(19) United States
(12) Patent Application Publication
(10) Pub. No.: US 2004/0202699 A1
(43) Pub. Date: Oct. 14, 2004

(54) Textile for weight reduction

(76) Inventor: Wu-Sung Kim, Busan-City (KR)

Correspondence Address: Ladas & Parry, Suite 1200, 224 South Michigan Avenue, Chicago, IL, 60604, US

(21) Appl. No.: 10/448,572

(22) Filed: May 30, 2003

(30) Foreign Application Priority Data
Apr. 8, 2003 (KR) 10-2003-0022147

(51) Int. Cl. A61K 9/70; A61K 33/04

(52) U.S. Cl. 424/443; 424/702

(57) ABSTRACT

As explained above, if and when the Solitonium is used in the products intended for weight reduction in accordance to the present invention, to ensure beneficial life for the humankind, then fat can be decomposed from the above-mentioned Solitonium and pain may be mitigated as well.

### BACKGROUND OF THE INVENTION

1. Field of the Invention

The present invention pertains to the textile for weight reduction that contains Solitonium and its usage. In particular, the purpose of the present invention pertains to the supply of product for weight reduction that can decompose human fat based on the use of Solitonium.

2. Description of the Prior Art

Due to the recent increase in the consumption of nutrients and lack of exercise, the number of obese population is on the rise. Excessive weight is considered a key factor that induces all types of diseases that afflict adults.

Accordingly, diverse weight reduction methods are suggested. Among these, the method of discharging large amount of sweat to reduce weight is most preferred. In order to discharge large amount of sweat, considerable amount of exercise is required. Therefore, ‘sweat wear’, which is manufactured with the materials that do not transmit air or moisture, is manufactured for sales, enabling discharge of large amount of sweat with low amount of exercise.

However, hitherto sweat wears lack the ability to exert pressure on the body since they are produced, using vinyl textile. Therefore, body heat is discharged through loose parts, which leads to low discharge of sweat. Moreover, hitherto sweat wears convey uncomfortable feeling since the vinyl textile cannot absorb sweat, and could induce allergy by coming into contact with the skin.

Furthermore, sweat wears that utilize vinyl material need to be manufactured in a form that the entire body is covered to achieve specific level of effect in discharging sweat. Therefore, the above-mentioned wears face difficulty in being produced as swimsuits, similar one-piece garments or inner wears such as brassieres. Accordingly, they are difficult to be worn frequently.

Moreover, synthetic textiles such as nylon and polyester that are used frequently for general sports wear textiles do not have the ability to absorb sweat due to their inherent characteristics. Therefore, they are marked with the weakness of not being able to absorb sweat when worn as garment.

To solve the above-mentioned problems, technology that enables breathable into the textile by leveraging methods such as drying coating, humidifying coating or laminating were applied. However, breathing function alone cannot satisfy 100% refreshing feel to the human body at the time when sweat is discharge in large amount in a short period of time.

In detail, hitherto technology that enables the ability to the synthetic textiles use the method of squeezing at a specific pick up rate (60˜100%) after padding the synthetic textile inside the hydrophile property absorption solution. The method mentioned above is not appropriate for sports wear textile since the ability of the textile to water-proof and to prevent the water from the outside to infiltrate the ears since the ability to absorb is enables both at the surface and back side of textile.

Moreover, other hitherto technologies coat the polyurethane resin situated on the synthetic textile by using drying, humidifying or laminating methods. Therefore, a method of enabling breathable is utilized to discharge the sweat generated from the human body, even if partially, instead of enabling the ability to absorb. However, the above-mentioned method cannot exert the level of absorption that is comparable to that of natural textile such as cotton. Therefore, the refreshing feeling is decreased when the sweat is discharged in large amount in an intensive period of time.

### SUMMARY OF THE INVENTION

The present invention is the culmination of years of research and experimentation, completed by focusing on the effect of Solitonium on the resourceful daily life of the human kind. When it pertains to Neoprene, when the above-mentioned Solitonium is adhered on and used on products intended for weight reduction, the purpose of the present invention is to provide textile for weight reduction that contains Solitonium, which enables decomposition of fat from the Solitonium and its usage.

When it pertains to the textile for weight reduction, the present invention contains Neoprene sponge layer that contains Solitonium; and Solitonium that contains coated endoderm.

### BRIEF DESCRIPTION OF THE DRAWINGS

The aforementioned aspects and other features of the present invention will be explained in the following description, taken in conjunction with the accompanying drawings, wherein:

FIG. 1 is the expanded figure of the structure of the textile for weight reduction that contains Solitonium in accordance to the present invention,

FIG. 2a˜FIG. 2d demonstrates the usage state of various products that utilize textile that contains Solitonium in accordance to the invention,

FIG. 3 is the figure that demonstrates the example on the diet garment for weight reduction that contains Solitonium in accordance to the present invention.

### EXPLANATION OF SYMBOLS PERTAINING TO KEY PARTS OF FIGURES

10—Products that Use Textile for Weight Reduction that Contains Solitonium

12—Neoprene sponge layer

14—endoderm

### DETAILED DESCRIPTION OF PREFERRED EMBODIMENTS

The present invention focuses on the fact that effective function of Solitonium, a new material, on the daily life of the humankind, and which is different from the hitherto garments intended for weight reduction, mentioned above. The present invention is developed based on many years of research and experimentation. When it pertains to Neoprene, when the above-mentioned Solitonium is adhered on and used on products intended for weight reduction, the purpose of the present invention is to provide textile for weight reduction that contains Solitonium, which enables decomposition of fat from the Solitonium and its usage.

Composition of the Invention:

The present invention, which intends to attain the above-mentioned purpose, is characterized by the inclusion of Neoprene sponge layer that contains Solitonium that contains Solitonium, coated with endoderm when it pertains to the textile for weight reduction.

Here, the above-mentioned Solitonium is the textile for weight reduction that is characterized by the following proportion of elements.

| Category of Sample | Solitonium Powder |
|---|---|
| SiO₂ | 75.405 |
| Al₂O₃ | 13.802 |
| Fe₂O₃ | 2.594 |
| MnO | 0.067 |
| CaO | 1.006 |
| MgO | 0.507 |
| K₂O | 1.416 |
| Na₂O | 0.87 |
| P₂O₅ | 0.081 |
| TiO₂ | 0.245 |
| LOI | 1.001 |
| TOtal | 96.994 |

<bead quantitative analysis (%)>

The above-mentioned element is the “data from the XRF analysis” that was measured with the method of “bead quantitative analysis”. The analysis was conducted at the “KOREA BASIC SCIENCE INSTITUTE”, using the “PHILIPS PW2400” measurement device (Unit: weight %). LOI stands for “Loss of Ignition” and generally represents the amount lost at about 950 C degree.

The products that utilize the above-mentioned textile for weight reduction that contains Solitonium include: abdominal support, garment for parts, diet wear, support for wrist, heels and elbow, diet garment, golf glove, wrist belt and bend. Moreover, the products include numerous sports goods that can be applied onto the human body, and which are not mentioned hereto.

More detailed explanation on the actual effect of the present invention shall be provided below by using attached figures as reference.

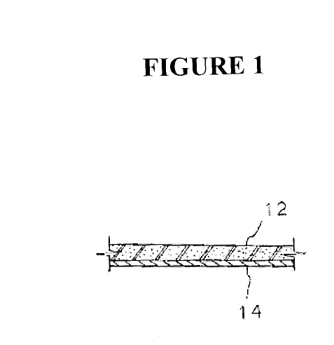

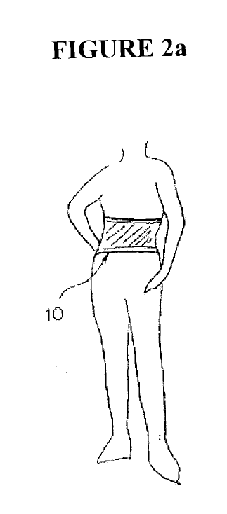

FIG. 1 is the expanded figure that demonstrates the structure of the textile for weight reduction that contains Solitonium in accordance to the present invention.

As shown on FIG. 1, textile for weight reduction that contains Solitonium contains Neoprene sponge layer(12) that contains Solitonium, and endoderm(14) with Solitonium coated towards the inner side of the above-mentioned Neoprene sponge layer(12) in accordance to the present invention.

The above-mentioned Neoprene is known as the source that is often used in sports wear. The present invention contains Solitonium within the above-mentioned Neoprene. Moreover, by coating Solitonium onto the endoderm, the present invention enables the optimization of fat decomposition. The following explains the effect of the above-mentioned Solitonium on the human body.

First of all, as if well established, Soliton is the solitary wave that behaves like particle. Likewise, Soliton is nonlinear wave that is transmitted in safe lump since the energy is concentrated. When the above-mentioned Soliton collide with each other, Soliton does not disintegrate. Instead, they let each other pass, and they do not disperse significantly, but are transmitted as is even when irregularities or alien materials are interposed on the medium. The above-mentioned is stable in this respect. If and when wave occurs within the ditch or canal that is of specific depth of h, the shape of the ridge of single wave does not change. Instead, transmission takes place at a consistent speed. The above-mentioned phenomenon was discovered experimentally by J. S. Russell of the U.K. in 1844, who in turn denominated the above-mentioned as the Soliton.

According to the Rayergh's theory, the approximation of the wave type that is the height of the wave q that proceeds towards the X direction and speed of c is,

η=a sec h2(x/2b) \mathring{1}

The above-mentioned is described as c2=g(h+a)b2=h2(h+a)/3a. However, a is the maximum value of A, and g is gravity's degree of acceleration. Wave equation of 1 dimension is,

$$\frac{{\partial }^{2} η}{\partial {t}^{2}} = {c}^{2} \frac{{\partial }^{2} η}{\partial {t}^{2}} \qquad 2 ◯$$

and is, η=A cos k(x−ct) or A sin k(x−ct),k 2πc/k. Wave equation of the above-mentioned shallow water wave is,

$$\frac{{\partial }^{2} η}{\partial {t}^{2}} = gh \{ \frac{{\partial }^{2} η}{\partial {x}^{2}} + \frac{3}{2 h} \frac{{\partial }^{2} ( {η}^{2} )}{\partial {x}^{2}} + \frac{{h}^{2}}{3} \frac{{\partial }^{4} η}{\partial {x}^{4}} \} \qquad 3 ◯$$

When \mathring{1} and \mathring{3} are compared, Clause 2 and 3 of the \mathring{3} do not exist. In case of h>>a, c2=gh occurs and \mathring{2} and \mathring{3} coincide. In other words, Clause 2 and 3 are nonlinear wave equation that is like \mathring{3} is found to correlate with, KdV equation (Kordeweg-de Vries equation)

ρt−6ηηx+ηxxx=0 \mathring{4}

However, t and x signify differential. Theories pertaining to numerous physics related phenomena such as plasma laser and electric wave of Defects in Crystals and others are unfolding newly from the above-mentioned Soliton point of view.

To gain a more accurate understanding of the above-mentioned Soliton, theory of material wave is necessary. The following is a summary of the theory on the material wave.

First of all, the power that governs the universe is the universal gravitation. Since Isaac Newton, scientists provided explanations on the power that mutually pulls the movement of planet, star and galaxy, in other worlds, the universal gravitation. However, contrary to the gravitation that pulls each other, repulsive force, which the power that pushes away each other, is reacting strongly in the universe. The main character of hereby mentioned repulsive force is the “vacuum energy”, which reacts strongly against ⅔ of the energy found in the entire universe (share of energy material in the universe is approximately 35%, and the remaining is the vacuum energy).

The above-mentioned vacuum energy generates exclusive “vacuum energy” for all the target and form of life including the cells with the system of gyration. And the self structure and state maybe changed due to the “vacuum energy” of the outside that influences life force.

Hereby mentioned vacuum energy is generated by the gyration of electronics, which is the basic mechanism pertaining to the generation of all “energy of the universe” that exists in the universe. Energy of the Universe is generated during the process of repeating +½−½ of the electronics' gyration, and the energy is generated forever since gyration movement takes places incessantly. Modification or copying the structure of water and all questions regarding all phenomena can unravel naturally once the hereby mentioned principle of energy of the universe is understood.

The above-mentioned energy of the universe is situated at the center of the generation and extinction of all creations. To put it easily, materials that are considered beneficial to our life contains helpful vacuum energy, and the materials that are known to be harmful to our body project out harmful vacuum energy into our body. The above-mentioned is the easy to understand explanation. Energy assumes on the form of wave. Energy of the Universe that is generated from the material is referred to as the material wave or wave of material.

Likewise, the wave material that is helpful to the human body may extinguish harmful waves (water vein wave, electronics energy wave, electromagnetic waves etc.). The Law of Immutable Energy does not apply in the face of the Principle of Energy of the Universe since Energy of the Universe occurs infinitely in a natural manner.

The principle on the fat decomposition that utilize material wave is as follows. All harmful phenomena that exist in the universe or in the world of human beings may be extinguished by the Energy of the Universe, that is, the beneficial material wave. In other words, phenomenon, that is, the state may be changed, as mentioned above. The human body is like the universe, which makes it extremely complicated and profound. Despite the level of complexity and profoundness, the above-mentioned eventually exist within the intervention of the wave of material.

Our body cannot help but react according to the type of energy (material wave) that is taken in by the body. In other words, when the human body is placed in front of beneficial wave, phenomena that were harmful to the human body up to date, due to the above-mentioned wave, are either changed for the better or simply undergo change.

1. Fat, accumulated in the human body, is taken in since the human body needs it, but when the fact is accumulated in excessive amount, the various types of phenomena are induced, which turn into disease. The principle of the universe is such that the harmful fat is eventually extinguished in the face of beneficial wave of material. This is indeed a mystery, but true.

2. Since the human body is immensely profound, it reacts promptly to all types of diseases. Reaction of fat decomposition is no exception, thus when fat is excessively accumulated in the human body, and then the body increases the rate of metabolism to consume greater-than-normal amount of energy.

The above-mentioned consumption of energy stimulates the muscle and may induce the generation of greater number of hormone through the movement that secretes Noradrenalin, Adrenaline, and Adrenocorticotropic Hormone etc from the terminal joints of the mutual response nerve. The above-mentioned is the reason that exercise is highly recommended. Moreover, in order to decompose the fat, extensive oxygen is needed, and this is the reason that cardio exercise is recommended.

However, the following explains how the material wave reacts in the human body and how it decomposes the fat.

As in the case of the above-mentioned Solitonium, the electromagnetic waves that are beneficial to the human body, that is, the material that discharges energy of the universe, need oxygen or at the presence of oxygen, it immediately changes all the materials that are situated in the area that transform them into beneficial material into the material with oxygen. When these materials are used to conduct BOD and COD tests, it is possible to confirm the above-mentioned easily.

The above-mentioned phenomenon is not chemical reaction, but a state in which the state changed immediately due to the energy of the universe. First of all, material wave requires large amount of energy by vitalizing all functions of the human body, which is considered a small scale universe. Even when the human beings are not exercising, large amount of exercise is taking place inside the human body. When the amount of blood flown and the speed of blood flow are checked, then these exercise phenomena become obvious. Metabolism rate is increased, and the increase in the metabolism rate results in the improvement of the energy consumption.

Increase in energy consumption leads to the increase in the generation of hormone, used for fat decomposition. At the end, fat is decomposed, and the goal that we demand is satisfied. Moreover, the material wave induces the improvement of the muscles through the vitalization of physiological activities of the human body. The hereby mentioned improvement in muscular strength signify strengthening of the red muscle, need for the endurance level. Therefore, the above-mentioned enables long exercises.

Diet shall be conducted in conjunction with cardio exercise, and the cardio exercise requires endurance level. Therefore, strengthening of muscles with endurance level is required material wave transforms the state of the user, which means that the former is already attaining the above-mentioned. This is the principle behind the fat composition of the human body by the Solitonium, which discharges the beneficial energy of the universe onto the human body.

As mentioned above, the above-mentioned Solitonium generates the material wave that moves while maintaining its shape like in the case of particle since the energy is concentrated, in other words, the Solitonium generates solitary wave. This in turn refers to the denomination of new material that transforms surrounding material into a positive material. After the mixing of purely natural mineral based composite, which has different wave characteristics, the rare-earth that underwent nano particle process is added on after dispersing to plasticity for a defined period of tome at a preset high temperature. The resulting material is the Solitonium that is a material for the transmission of the physiological activity vitalization wave that is characterized by the transformed structure of the combined crystalline.

Moreover, the above-mentioned characteristics of Solitonium, that is the Solitonium, which is the material for the transmission of physiological activity vitalization wave, is the mechanism that ideally controls the balance of the substance in the body based on the use of vitalization reaction of the cells situated in the body. As a consequence, the above-mentioned Solitonium acts as the outstanding vitalization agent of the human body due to acceleration of blood circulation, metabolism reaction, partial heating, irregularity blood pressure effect, control of irregular electric potential within the body, pain mitigation due to electric discharge reaction, increase in immunity capability etc.

Accordingly, the key functions of the Solitonium are as follows:

1. Pain mitigation,

2. fat decomposition reaction and control of balance within the substance in the body,

3. increase in immunity capability of the body and catalyzing of growth hormone,

4. catalyzing reaction of metabolism and improvement of blood circulation,

5. improvement of problems in blood flow, high hemorrhage, diabetes, high blood pressure etc,

6. decrease in the level of fatigue during and after exercise,

7. antibiotic function, and

8. harmful electromagnetic waves and effect of blocking water vein, and others.

The data pertaining to the test of the above explained Solitonium by the “KOREA BASIC SCIENCE INSTITUTE” is as follows:

[image]

According to the analysis report of the above-mentioned “KOREA BASIC SCIENCE INSTITUTE”, blood vessels are classified into two groups; Twelve Regular Meridians and, eight extra meridians. Twelve Regular Meridians oversee our physical aspect, and the eight extra meridians are mostly responsible for the mental aspect. If and when the vital energy of these blood vessels is well operated, then we can maintain healthy body and mind. The flow of small group vital energy circulates each part and the five viscera and the six entrails (of gall bladder, stomach, small and large intestines, the paunch, the bladder and the bowels) of the human body and it flows along the Twelve Regular Meridians. If and when the small group vital energy does not circulate the Twelve Regular Meridians effectively, then we suffer from mental disease. Accordingly, the above-mentioned data proves that the Solitonium is an outstanding medium for circulating the vital energy of the blood vessel, more so than any other treatment medicinal materials.

The above-mentioned Solitonium is adhered onto the functional Neoprene material to produce textile for weight reduction and related products in accordance to the present invention. The above-mentioned Neoprene is the cutting edge adhesion technology that applies natural dyeing method. When the Solitonium is adhered on the Neoprene material, then the realization of functional Neoprene material that is endowed on the semi permanent function of body vitalization within the cleansing effect.

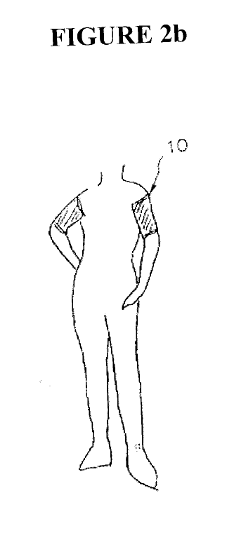

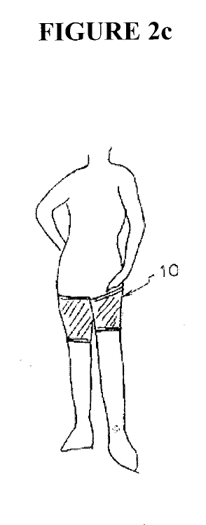

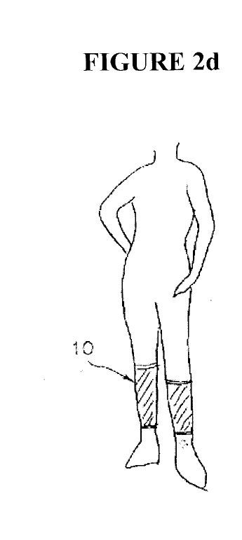

Likewise, Neoprene that contains Solitonium is used to produce product for weight reduction. The state of wearing the above-mentioned are shown on FIG. 2a˜FIG. 2d.

FIG. 2a˜FIG. 2d are the figures that show the state of using various products that utilize textiles that contain Solitonium according to the present invention.

Product(10) as shown on FIG. 2a is the abdominal support that may reduce fat in the waist and the lower part of the abdominal muscle in the human body. The above-mentioned abdominal support is made from the textile that contains the Solitonium, a material for the transmission of wave resulting from physiological activities as explained hereto. The above-mentioned vitalizes the cell in the body that is focused on the control the balance of the substance in the body. At the same time, the above-mentioned is designed in a way that it can exert outstanding function on the decomposition of fat on the abnormal abdomen.

The advantage of the above-mentioned abdominal support is that it decomposes only the excessive fat, which does not entail simple weight reduction, caused by the decrease in flesh or muscle. Accordingly, the above-mentioned induces balance within the body. Therefore, the above-mentioned transforms the body figure before the weight reduction. Accordingly, when used for one month, waist for the pants or skirts becomes loose. As a consequence, there are no negative symptoms such as decreased muscular strengths due to excessive weight reduction, sense of helplessness due to energy loss, dizziness, weakening of kidney functions and others. In fact, user can feel that all the functions of the body undergo improvement.

Moreover, the above-mentioned method is ideal for the exercise treatment method and for the control of balance within the substance in the body. There is no yoyo effect resulting from fat decomposition and weight loss. After weight loss, muscular power and endurance level increase. Furthermore, the above-mentioned is highly effective for improving problems in blood flow, high hemorrhage, diabetes, high blood pressure etc, caused by obesity.

The above-mentioned abdominal support is semi permanent and the used textiles do not emit smell of sweat due to its outstanding antibacterial and elimination of smell effect.

Moreover, at the interior of the above-mentioned abdominal support, heating element may be built in. At this time, discharge of heat is based on the facial heating element that uses direct current of 12V so that the generation of electromagnetic waves is precluded. As for the temperature of the heat subject to emission, maximum of 60 □ is recommended. In other words, it is well known that electromagnetic waves are not generated at direct current power.

Likewise, when the heating element is built into the abdominal support, decomposition of abdominal fat takes place by using the wave material of the Solitonium. Usage is enabled by wearing the above-mentioned after connecting to the power, which starts off the physiological activity reaction and fat decomposition. Accordingly, the above-mentioned maybe used easily while watching TV or reading at home and while sleeping. In particular, when worn at the fitness club or while jogging, the effect of fat composition becomes greater. Moreover, the above-mentioned abdominal support should be accompanied by the following recommended diet—three meals per day is recommended, but excessive eating or consumption of unnecessary snacks and the consumption of overly fatty food is discouraged. Moreover, drinking is discouraged as well. In particular, snacking after the dinner is very harmful, and daily light jogging or walking are essential.

Comparative Test for Before and After Use of the “Slim Device”
Based on the BRS Analysis Device for the Measurement of Human
Body Information

| | | Before | After 8 Hrs | |
|---|---|---|---|---|
| ITEM LIST | | BRS Result | BRS Result | Ratio (%) |
| 1. Immunity Function, | B222 | +8 | +11 | 37.5 |
| 2. Bladder, | 36FD | +5 | +8 | 60.0 |
| 3. Lung, | D996 | +9 | +10 | 11.1 |
| 4. Heart, | D166 | +4 | +7 | 75.0 |
| 5. Kidney, | D802 | +6 | +10 | 66.6 |
| 6. Liver, | D273 | +7 | +9 | 28.5 |
| 7. Pain, | D468 | +7 | +10 | 42.8 |
| 8. Toxin for Fatigue, | D684 | +6 | +10 | 66.6 |
| 9. Large Intestine, | D157 | +5 | +9 | 80.0 |
| 10. Cold-type infantile | 1061 | +8 | +10 | 25.0 |
| malnutrition of Limbs, | | | | |
| 11. Small Stomach, | E240 | +8 | +10 | 25.0 |
| 12. Stomach/duodenum, | D199 | +5 | +10 | 100.0 |
| 13. Uterus | D449 | +7 | +10 | 42.8 |

[symbol]

:

[symbol]

The test data pertaining to the before and after use of the above-mentioned abdominal support is as follows.

The above-mentioned data is the comparative test data pertaining to the use of the above-mentioned abdominal support. The test is based on the use of INBODY 3.0 fat analysis device.

[image]

The above-mentioned data is the data resulting form the comparative test of before and after using the above-mentioned the above-mentioned abdominal support. Test was conducted based on the use of measurement device for the analysis of BRS human body information.

As can be witnessed from the above-mentioned data, when the above-mentioned abdominal support is worn, then the effect of fat decomposition is outstanding.

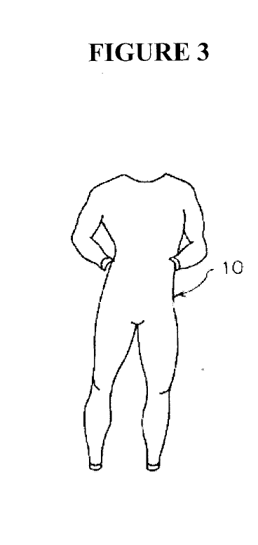

FIG. 2b˜FIG. 2d show that the present invention maybe worn in different parts of the human body. Product (10), depicted on FIG. 2b is the band used for the elbow or the area between the shoulders. The Product (10), shown on FIG. 2c is the band that is used on the part pertaining to the flesh inside the thigh. Product (10), shown on the FIG. 3d is the band that is used on the part pertaining to the calf of the leg. The same effect as the abdominal support mentioned above can be obtained. Moreover, products that can be worn on the wrist that is not shown hereto and other diverse products such as golf glove may be obtained. The composition of all these products is already well known, and there is a difference in the materials used for the production of the above-mentioned products (10) when it pertains to the present invention.

FIG. 3 is an example of the garment for diet used for weight reduction that contains Solitonium in accordance to the present invention.

Product (10), depicted on FIG. 3, is the is wear that can be worn in all parts of the human body except for the face so that entire part of the human body can benefit from fat reduction and weight reduction effects. The effects mentioned above may result. Moreover, all products pertaining to the present invention can mitigate pain as well.

1. A textile for weight reduction comprising:
a Neoprene sponge layer containing a Solitonium; and
an endoderm layer coated with the Solitonium on one side of the endoderm layer joining the Neoprene layer,
wherein the elements of the Solitonium analyzed by a quantitative analysis consist essentially of:
SiO₂ of about 75% by weight;
Al₂O₃ of about 13% by weight;
Fe₂O₃ of about 3% by weight;
MnO of less than 0.1% by weight;
CaO of about 1% by weight;
MgO of about 0.5% by weight;
K₂O of about 1% by weight;
Na₂O of about 1% by weight;
P₂O₅ of less than 0.1% by weight; and
TiO₂ of less than 0.3% by weight; and
further wherein about 1% of the total weight of the Solitorilum is lost at about 950 C degree during the quantitative analysis.

2-4. (cancelled)

5. The textile for weight reduction of claim 1, wherein the textile for weight reduction containing the Solitonium is capable of being used in clothing articles comprising:
an abdominal support;
an article to be worn on different parts of the body, each part of the body including an elbow, a shoulder, a thigh, a leg calf, and a wrist;
a diet garment that is worn by the most parts of the body including the arms, the legs, and the torso of the body; and
a golf glove.

6. (cancelled)

7. A clothing article containing a textile for weight reduction, the textile comprising a Neoprene sponge layer containing a Solitonium and an endoderm layer coated with the Solitonium on one side of the endoderm layer joining the Neoprene layer,
wherein the elements of the Solitonium analyzed by a quantitative analysis consist essentially of;
SiO₂ of about 75% b eight;
Al₂O₃ of about 13% by weight;
Fe₂O₃ of about 3% by weight;
MnO of less than 0.1% by weight;
CaO of about 1% by weight;
MgO of about 0.5% by weight;
K₂O of about 1% by weight;
Na₂O of about 1% by weight,
P₂O₅ of less than 0.1% by weight; and
TiO₂ of less than 0.3% by weirht and
further wherein about 1% of the total weight of the Soliton urn is lost at about 950 C degree during the quantitative analysis.

8-10. (cancelled)

11. The clothing article of claim 7, wherein the clothing article comprising:
an abdominal support;
an article to be worn on different parts of the body, each part of the body including an elbow, a shoulder, a thigh, a leg calf, and a wrist;
a diet garment that is worn by the most parts of the body including the arms, the legs, and the torso of the body; and
a golf glove,

12. (cancelled)
